(19) United States
(12) Patent Application Publication
(10) Pub. No.: US 2017/0137396 A1
(43) Pub. Date: May 18, 2017

(54) PROCESS FOR THE SELECTIVE OXIDATION OF 5-HYDROXYMETHYLFURFURAL

(71) Applicants: ANNIKKI GMBH, Graz (AT); MICROINNOVA ENGINEERING GMBH, Allerheiligen b. Wildon (AT)

(72) Inventors: Marko Mihovilovic, Perchtoldsdorf (AT); Michael Schön, Wien (AT); Stefan Dochev, Sofia (BG)

(21) Appl. No.: 15/319,065

(22) PCT Filed: Jun. 17, 2015
(86) PCT No.: PCT/EP2015/063578

§ 371 Date: Dec. 15, 2016

(30) Foreign Application Priority Data
Jun. 17, 2014 (EP) 14172678.6

(51) Int. Cl. C07D 307/46

(52) U.S. Cl. CPC C07D 307/46

(57) ABSTRACT

Process for the selective production of oxidized furan derivatives starting from 5-hydroxymethyl-2-furfural in the presence of a solvent, an oxidation agent, a catalyst, and optionally a base, which process is characterized in that the oxidation process is carried out continuously in flow, and there are provided means for varying reaction parameters.

The present invention relates to selective oxidation of 5-hydroxymethylfurfural. 5-Hydroxymethyl-2-furfural (HMF) of formula

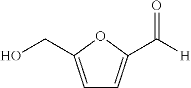

plays an important role in renewable carbohydrate technology and reflects a central intermediate in furan chemistry. Triple carbohydrate monomer dehydration of sugars leads to the formation of HMF, which is widely known in literature. HMF provides three sites of chemical interest—the 5-hydroxymethyl group, the 2-carbaldehyde group and the furan ring itself. By far of highest interest to industry are the two side chains, which can be oxidized to obtain various furan derivatives.

According to the present invention, the four oxidized HMF derivatives 5-hydroxymethylfuran-2-carboxylic acid (HMFCA) of formula

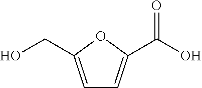

2,5-diformylfuran (DFF) of formula

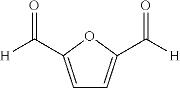

5-formylfuran-2-carboxylic acid (FFCA) of formula

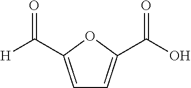

and

2,5-furandicarboxylic acid (FDCA) of formula

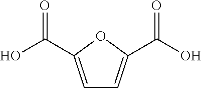 [SMILES: O=C(O)c1ccc(C(=O)O)o1]

are of particular interest.

HMFCA may be regarded as a result of selective oxidation of the aldehyde group in HMF to obtain the carboxylic acid. For such selective oxidation, only a small number of protocols are known. In most of the cases, expensive silver-based reagents are used in stoichiometric amount to synthesize HMFCA. Silver oxide in basic (NaOH) aqueous medium (Bull. Soc. Chim. Fr. 1987, 5, 855-860) as well as mixed silver-copper catalysts Ag₂O—CuO/O₂/NaOH/H₂O (U.S. Pat. No. 3,326,944, 1967) are the most commonly used reagents. Economically, these reagents cannot be applied on large industrial scale. Therefore, precious metal catalysts (especially platinum catalysts) were proposed, e.g. as described in ChemSusChem 2009, 2, 1138-1144; ChemSusChem 2009, 2, 672-675; Catal. Today 2011, 160, 55-60; Green Chem. 2011, 13, 824-827; Green Chem. 2011, 13, 2091-2099) or ruthenium-based catalysis (Top Catal. 2011, 54, 1318-1324; Catal. Lett. 2011, 141, 1752-1760). The oxidation process was mainly carried out in the presence of air and in aqueous reaction environment to synthesize HMFCA in good yield and with high turnover frequency (TOF) rendering the process economically and environmentally benign.

In the synthesis of DFF, a larger number of protocols is known. In batch synthesis, classical oxidation reactions using nitric acid (J. Chem. Soc. Trans. 1912, 101, 1074-1081), lead-(IV)-acetate/pyridine (Tetrahedron 1970, 26, 1291-1301), CrO₃/pyridine or Ac₂O/DMSO (Noguchi Kenkyusho Jiho 1978, 21, 25-33; JP7909260, 1979; JP8049368, 1980), BaMnO₄/benzene/CCl₄/1,2-dichloroethane (Bull. Soc. Chim. Fr. 1987, 5, 855-860; J. Heterocycl. Chem. 1983, 20, 233-235) or 4-substituted TEMPO/NaOCl/KBr (J. Heterocycl. Chem. 1995, 32, 927-930) are known.

Taking benefit of catalysis, extensive research was already carried out using homogeneous and heterogeneous catalysis. DFF could be synthesized in batch using cobalt, manganese, zinc, cerium or zirconium salts together with a gaseous oxidant (US 2003/055271 A1, 2003; Adv. Synth. Catal. 2001, 343, 102-111; WO 01/072732 A2, 2001; CA2400165 A1, 2001; WO 2010/132740 A2, 2010; Catal. Sci. Technol. 2012, 2, 79-81). Furthermore, also diverse vanadium catalysts were reported (ChemSusChem 2011, 4, 51-54; Green Chem. 2011, 13, 554-557; J. Mater. Chem. 2012, 22, 3457-3461). In the heterogeneous catalysis, mainly vanadium—(Pure Appl. Chem. 2012, 84, 765-777; ChemCatChem 2013, 5, 284-293), manganese—(Green Chem. 2012, 14, 2986-2989) and silver-based catalysts (WO 2012/073251 A1, 2012; Appl. Catal. B 2014, 147, 293-301) were applied in organic solvents.

Technologically different, also the approaches of sonochemistry (Org. Prep. Proced. Int. 1995, 27, 564-566; Pol. J. Chem. 1994, 68, 693-698) and electrochemistry (Synthesis 1996, 11, 1291-1292) were followed - both of inferior interest for selective, large scale processes on industrial scale.

Although many publications dedicated to the selective oxidation of HMF to DFF are published in literature, only a limited number of described conditions could potentially find industrial application, meeting the requirements for safe, fast, environmentally and economically benign processes. However, reported processes rely on the use of organic solvents, which are troublesome when used in combination with powerful, pressurized oxidants such as pure oxygen. In addition, continuous flow technology was only used so far with a quite specific reaction strategy, wherein a hypervalent iodine species (BAIB) or HNO₃ were used in combination with catalytic amounts of TEMPO (Beilstein J. Org. Chem. 2013, 9, 1437-1442; Green Chem. 2013, 15, 1975-1980).

A further oxidized derivative of HMF is FFCA, which due to its high reactivity and instability is only poorly reported in literature. It can be synthesized using complex catalytic systems such as 4-BzOTEMPO/acetylcholine chloride/Py*HBr₃ in biphasic reaction medium (Bull. Chem. Soc. Jpn. 2009, 82, 1000-1002), strongly acidic conditions under gold catalysis (Catal. Sci. Technol. 2012, 2, 79-81) or precious metal catalysis in flow, but without precise determination of residence times and space-time-yields rendering the process less attractive for cost-efficient production of FFCA (Top Catal. 2010, 53, 1264-1269).

FDCA was also reported as an oxidized furan derivative of particular interest, due to its potential application as replacement for terephthalic acid in polyester synthesis. Also here, classical oxidation was carried out using nitric acid (Chem. Weekblad 1910, 6, 717-727; Noguchi Kenkyusho Jiho 1979, 22, 20-27; Pol. J. Chem. 1994, 68, 693-698) or permanganate (Bull. Soc. Chim. Fr. 1987, 5, 855-860) to selectively give FDCA as product. In the field of catalytic processes, homogeneous catalysts from the cobalt-, manganese-, zinc-, cerium- and zirconium-type are readily known (US 2003/055271 A1, 2003; Adv. Synth. Catal. 2001, 343, 102-111; WO 01/72732 A2, CA2400165 A1, 2001; WO 2010/132740 A2, 2010; US 2009/0156841 A1, 2009; WO 2011/043661 A1(A2), 2011; Catal. Sci. Technol. 2012, 2, 79-81; WO 2012/161967 A1, WO 2012/161970 A2; US20120302769 A1, 2012).

Using heterogeneous catalysis, gold (ChemSusChem 2009, 2, 1138-1144; ChemSusChem 2009, 2, 672-675; Top Catal. 2012, 55, 24-32), ruthenium (Top Catal. 2011, 54, 1318-1324; Catal. Lett. 2011, 141, 1752-1760) as well as platinum catalysts (Top. Catal. 2000, 13, 237-242; U.S. Pat. No. 3,326,944, 1967; Stud. Surf. Sci. Catal. 1990, 55, 147-157; Stud. Surf. Sci. Catal. 1991, 59, 385-394; Top Catal. 2010, 53, 1264-1269) were used, eventually also in flow.

Further processes involving reaction of HMF into oxidation products are known from WO 2012/017052 A1 and WO 2008/054804 A2.

However, summarizing the process parameters and characteristics, no precisely determined, environmentally and economically benign, intrinsically safe and scalable process for the modular synthesis of HMFCA, DFF, FFCA and FDCA has been reported yet.

Now, surprisingly a process for the production of different oxidized 5-hydroxymethylfurfural derivatives, such as 5-hydroxymethylfuran-2-carboxylic acid (HMFCA), 2,5-diformylfuran (DFF), 5-formylfuran-2-carboxylic acid (FFCA) and 2,5-furandicarboxylic acid (FDCA) from HMF in the same reactor setup was found.

In one aspect, the present invention provides a process for the selective production of oxidized furan derivatives starting from 5-hydroxymethyl-2-furfural of formula

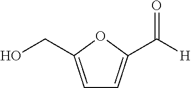

in the presence of a solvent, an oxidation agent, a catalyst, and optionally a base and/or a co-solvent, which is characterized in that
the oxidation process is carried out continuously in flow,
there are provided means for varying reaction parameters, such as temperature, pressure, oxidation agent, and/or catalyst.

A process provided by the present invention is also designated herein as “Process(es)” according to the present invention.

Preferably, in the process of the present invention the solvent for the oxidation process is water and a dipolar aprotic solvent is present as a co-solvent. Especially preferably N-methylpyrrolidone is present as a co-solvent.

Oxidized furan derivatives in a process of the present invention comprise at least one aldehyde group and/or at least one carboxylic acid group, preferably 5-hydroxymethylfuran-2-carboxylic acid (HMFCA), 2,5-diformylfuran (DFF), 5-formylfuran-2-carboxylic acid (FFCA) and 2,5-furandicarboxylic acid (FDCA).

A process of the present invention is carried out in a solvent, preferably in water. Optionally a co-solvent may be present. Such co-solvent may be useful for better solubility or enables the use of an enriched HMF stream from previous dehydration reactions as a starting material. Typical examples for co-solvents are dipolar aprotic solvents, such as N,N-dimethylformamide, dimethylsulfoxide, N-methylpyrrolidone; preferably N-methylpyrrolidone.

A process for the production of HMF from carbohydrates, especially fructose, involving the use of NMP as a solvent is disclosed in WO 2014/033289. It has been found that it is possible to perform the process of the present invention using the HMF-enriched product stream, including NMP, of a process as disclosed in WO 2014/033289. Thus, there is no need to remove the NMP contained in said HMF-enriched stream before the oxidation step.

Accordingly, in one further preferred embodiment of the present invention, a stream enriched with 5-hydroxymethyl-2-furfural from previous dehydration reactions, in particular dehydrations of sugars, is employed as a starting material. In this embodiment, preferably a stream containing NMP as a solvent is employed and the process does not include a step of removing NMP before the oxidation step.

In this embodiment of the present invention, optionally before the oxidation step one or more pretreatment steps selected from

(i) real stream dilution with water to the desired concentration

(ii) centrifugation in order to separate any black tar formed during the preparation of the stream

(iii) filtration

(iv) passing the solution through a packed-bed cartridge filled with activated charcoal

may be carried out.

Furthermore, generally it has been found that dipolar aprotic solvents, including NMP, have advantageous properties especially in the oxidation of HMF to polar products such as FDCA, in terms of the homogenisation of the reaction mixture.

Finally, a positive influence of dipolar aprotic solvents, including NMP, on the stability of the catalysts (protection against deactivation) has been observed,

A process according to the present invention is carried out at a reaction temperature from 50° C. to 180° C., preferably from 60° C. to 160° C.

In a process of the present invention the reaction temperature for the production of
5-hydroxymethylfuran-2-carboxylic acid is from 60° C. to 120° C., in particular from 80° C. to 120° C., in particular from 100 to 120° C.;
2,5-diformylfuran is from 100 to 160° C., in particular from 120-160° C., in particular from 140° C. to 160° C.;
5-formylfuran-2-carboxylic acid is from 60° C. to 160° C., in particular from 80° C. to 140° C., in particular from 100° C. to 120° C.;
2,5-furandicarboxylic acid is from 60° C. to 160° C., in particular from 60° C. to 120° C., in particular from 80° C. to 120° C.

It has been found that when, in the process according to the invention, water is employed as a solvent and NMP is used as a co-solvent, slightly harsher reaction conditions are advantageous, especially in case the desired oxidation product is FDCA. Temperatures ranging from 120° C. to 160° C., in particular 140° C. to 160° C. have been found to be advantageous.

A process according to the present invention is carried out in the presence of an oxidation agent. Such oxidation agent is preferably oxygen or air, in particular compressed oxygen or compressed air.

A process of the present invention is carried out under pressure. A preferred working pressure is from 5 bar to 100 bar, in particular from 10 bar to 80 bar.

In a process according to the present invention, a catalyst is used. Catalysts for the production of oxidation products of HMF are known. A preferred catalyst for the production of DFF in a process of the present invention is K-OMS-2; a preferred catalyst for the production of HMFCA, FFCA and FDCA is 10% Pt/C.

K-OMS-2 and its use in catalysis is known. “OMS-2” stands for cryptomelane type crystalline mixed-valent manganese (oxide)-based octahedral molecular sieve(s). “K in K-OMS-2” stands for potassium. K-OMS-2 has approximately the molecular formula KMn₈O₁₆ having a 2×2 hollandite structure. “K-OMS-2” means that the pores (tunnels) of the OMS-2 are occupied by K⁺ ions, which neutralize the negative charge of the OMS-2 framework, consisting of edge- and corner-shared [MnO6]-octahedra.

In a process of the present invention for the production of HMFCA, FFCA and FDCA a base, e.g. a hydroxide, a carbonate or a bicarbonate, e.g. an alkali hydroxide, alkali carbonate or alkali bicarbonate, such as sodium hydroxide, sodium carbonate or sodium bicarbonate may be used as a co-catalyst as well as for increasing the solubility.

In a process of the present invention for the selective production of 2,5-furandicarboxylic acid starting from 5-hydroxymethyl-2-furfural, the combination of the following features has been found to be of particular advantage:
a base selected from the group of carbonates and bicarbonates, in particular sodium carbonate and/or sodium bicarbonate is used as a co-catalyst
the working pressure is from 80 to 100 bar.

This embodiment is especially preferred in case the oxidation agent is compressed oxygen. Especially, it has been found that in case of pressures lower than 80 bar deactivation of the catalysts employed was observed, leading to loss of yield in FDCA and loss of selectivity.

The preferred temperature in this embodiment of the present invention is from 120° C. to 160° C., more preferably from 140° C. to 160° C.

Further preferred, platinum on activated charcoal is used as the catalyst in this embodiment of the present invention.

Again, also in this embodiment, preferably water is used as a solvent. Furthermore, preferably a dipolar aprotic solvent, in particular NMP, is used as a co-solvent.

In contrast to known processes, the present invention provides a single process to synthesize four different furan derivatives of HMF using the same reactor setup just varying reaction parameters such as temperature, pressure, oxidation agent and/or catalyst. This reflects huge benefits in process optimization time, process costs and overall process efficiency impossible to achieve in batch chemistry.

Differently to existing batch protocols in which the reaction conditions need to be optimized from scratch, adapting reaction vessels to the chosen chemistry, the continuous-flow approach avoids these drawbacks in an elegant way. The most significant advantage of the developed process is the reduction of actual reaction volumes to very small volumes (usually lower than 1 mL), which also reduces the safety hazard by orders of magnitude. Even high pressures of pure oxygen can be safely handled and scaled as well—preferably by parallelization of continuous flow reactors rather than increasing reaction volumes.

In the following Reaction Scheme 1 oxidation reactions starting from HMF to obtain the four furan derivatives 5-hydroxymethylfuran-2-carboxylic acid (HMFCA), 2,5-diformylfuran (DFF), 5-formylfuran-2-carboxylic acid (FFCA) and 2,5-furandicarboxylic acid (FDCA) selectively in continuous flow according to the present invention are schematically outlined.

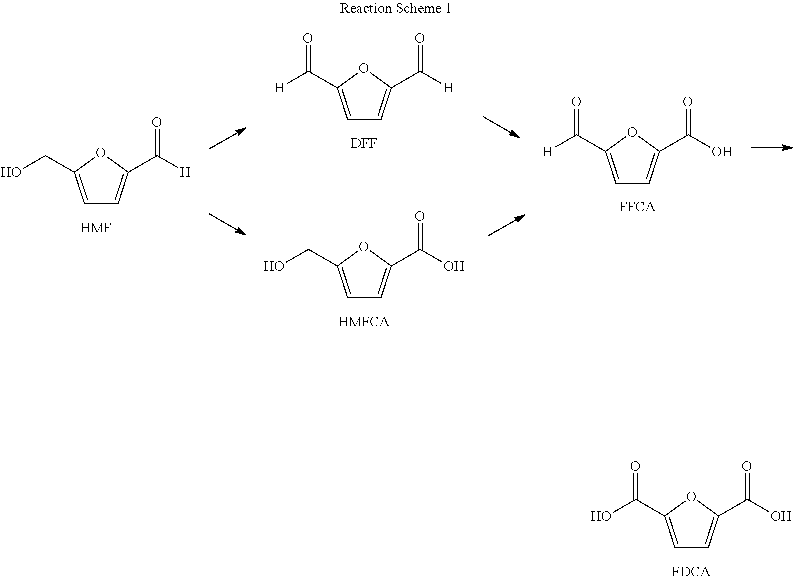

In the following examples all temperatures are in degrees Celsius (° C.),

The following abbreviations are used

DFF 2,5-diformylfuran

FDCA 2,5-furandicarboxylic acid

FFCA 5-formylfuran-2-carboxylic acid

HMF 5-hydroxymethyl-2-furfural

HMFCA 5-hydroxymethylfuran-2-carboxylic acid

HPLC high-performance (formerly high-pressure) liquid chromatography

K-OMS-2 manganese octahedral molecular sieve

min minutes

NMP N-methyl-2-pyrrolidone

PDA photo diode array

RI refractive index

T temperature

TFA trifluoroacetic acid

The yields in % in the Tables below are calculated based on the amount of the starting material HMF.

The reaction performance was evaluated in terms of HMF conversion and HMFCA, DFF, FFCA or FDCA yield/selectivity using HPLC (column: Phenomenex Rezex RHM 150×7.8 mm, mobile phase: 0.1 wt % TFA in H₂O, temperature: 85° C., flow rate: 0.6 mL/min, method duration: 23 min (NMP-free samples)/60 min (NMP-containing samples), detection: RI or PDA, internal standard: phenol).

### EXAMPLE 1

Oxidation of HMF to obtain HMFCA
Reactant HMF (5 mg/mL) in water
Base additive NaOH (2 equiv. based on HMF, mixed in situ with the solution of HMF via the second HPLC pump, supplied as 0.08 M solution in water)
Catalyst 10% Pt/C (280 mg 10% Pt/C+20 mg Celite 545)
Oxidant synthetic air
Reactor System ThalesNano X-Cube, pump flow rate: 2×0.5 mL/min, residence time: 1 min

Each CatCart (70×4 mm) was filled first with 20 mg of Celite 545 and then 280 mg 10% Pt/C were added. Fresh CatCart was used every time, when the system pressure was changed. Before each screening series, the entire reaction line was purged with H₂O (HPLC Grade), the Teflon frit of the system valve was replaced and ThalesNano X-Cube System Self-Test was performed. The initial system stabilization was always achieved using NaOH / H₂O solution and when the reaction parameters remained constant, the pumping of the reaction solution began, then the system was allowed to stabilize and equilibrate at the new conditions for 10 min and two samples of 1 mL each were then collected. Then the temperature was increased and the system was again allowed to stabilize (the same procedure was applied for all temperatures within the experimental series). In all the cases 40 bar difference between the system pressure and the external gas pressure was provided for good system stability. In the selective oxidation of HMF to HMFCA, temperature-mediated catalyst deactivation was used to synthesize HMFCA in favour of the fully oxidized FDCA.

Table 1 below provides a summary of the results from HMF-HMFCA oxidation screening in flow using the following parameters: 0.5 mL HMF (5 mg/mL), 0.5 mL NaOH (0.08 M), H₂O, 10% Pt/C, 80 bar Air, 60-120° C., 0.5 mL/min×0.5 mL/min, 1 min.

TABLE 1

| | | | | | | FDCA/ |
|---|---|---|---|---|---|---|
| | HMF | DFF | | | | HMFCA |
| T | conversion | yield | HMFCA | FFCA | FDCA | selectivity |
| [° C.] | [%] | [%] | yield [%] | yield [%] | yield [%] | [%] |
| 60 | 99.71 | 0.32 | 29.49 | 0.91 | 73.73 | 73.94/ |
| | | | | | | 29.58 |
| 80 | 99.71 | 0.32 | 30.66 | 1.26 | 66.39 | 66.58/ |
| | | | | | | 30.75 |
| 100 | 99.46 | 0.58 | 32.92 | 0.62 | 58.07 | 58.37/ |
| | | | | | | 33.10 |
| 120 | 95.57 | 0.32 | 80.65 | 3.07 | 20.15 | 21.09/ |
| | | | | | | 84.39 |

From Table 1 it is evident that with increasing temperature the HMFCA yield is increasing under the given conditions. The reaction preferably is carried out from 60° C. to 120° C., in particular from 80° C. to 120° C., in particular from 100 to 120° C. A sharp increase in HMFCA yield is obtained if the temperature exceeds 100° C. A particular preferred temperature is thus from 105 to 130° C., such as 110 to 125° C., e.g. 115 to 120° C.

### EXAMPLE 2

Oxidation of HMF to obtain DFF
Reactant HMF (5 mg/mL) in water
Catalyst K-OMS-2 (263.4 mg K-OMS-2+50 mg Celite 545) prepared according to Angew. Chem. Int. Ed. 2012, 51, 544-547.
Oxidant oxygen or synthetic air
Reactor System ThalesNano X-Cube, pump flow rate: 0.5 mL/min, residence time: 2/4 min

Each CatCart (70×4 mm) was filled first with 50 mg Celite 545 and then 263.4 mg K-OMS-2 were added. Fresh CatCart was used every time, when the system pressure was changed. Before each screening series, the entire reaction line was purged with H₂O (HPLC Grade), the Teflon frit of the system valve was replaced and ThalesNano X-Cube System Self-Test was performed. The initial system stabilization was always achieved using H₂O (HPLC Grade) and when the reaction parameters remained constant, the pumping of the reaction solution began, then the system was allowed to stabilize and equilibrate at the new conditions for 10 min and two samples of 1 mL each were then collected. Then the temperature was increased and the system was again allowed to stabilize (the same procedure was applied for all temperatures within the experimental series). In all the cases 40 bar difference between the system pressure and the external gas pressure was provided for good system stability.

The experiments were carried out using one or two catalyst cartridges offering ideal reaction conditions to produce DFF in good yield (˜70%) requiring only 10 bar of oxygen partial pressure.

To reduce the hazardous potential of pure oxygen, the reactions were also performed substituting oxygen with synthetic air. However, to reach similar yields, the pressure had to be increased to 80 bar of compressed air.

In Table 2 below there is set out a summary of the results from HMF-DFF oxidation screening in flow using the following parameters:

1 mL HMF (5 mg/mL), H₂O, K-OMS-2/Celite, 10 bar O₂, 100-160° C., 0.5 mL/min, 2 min (using one catalyst cartridge).

TABLE 2

| | HMF | DFF | DFF | | | FDCA |
|---|---|---|---|---|---|---|
| T | conversion | yield | selectivity | HMFCA | FFCA | yield |
| [° C.] | [%] | [%] | [%] | yield [%] | yield [%] | [%] |
| 100° C. | 30.97 | 20.24 | 65.47 | 0.00 | 4.63 | 0.15 |
| 110° C. | 40.80 | 28.51 | 70.19 | 0.00 | 3.01 | 0.00 |
| 120° C. | 49.97 | 37.13 | 74.51 | 0.00 | 4.77 | 0.00 |
| 130° C. | 61.42 | 48.43 | 79.06 | 0.00 | 7.44 | 0.00 |
| 140° C. | 73.19 | 54.23 | 74.08 | 0.00 | 10.09 | 0.00 |
| 150° C. | 82.76 | 63.16 | 76.32 | 0.00 | 12.83 | 0.26 |
| 160° C. | 88.74 | 69.00 | 77.88 | 0.00 | 14.55 | 0.89 |

In Table 3 below there is set out a summary of the results from HMF-DFF oxidation screening in flow using the following parameters:

1 mL HMF (5 mg/mL), H₂O, 2× K-OMS-2/Celite, 10 bar O₂, 100-160° C., 0.5 mL/min, 4 min (using two catalyst cartridges)

TABLE 3

| | HMF | DFF | DFF | | | |
|---|---|---|---|---|---|---|
| T | conversion | yield | selectivity | HMFCA | FFCA | FDCA |
| [° C.] | [%] | [%] | [%] | yield [%] | yield [%] | yield [%] |
| 100 | 47.91 | 35.48 | 74.09 | 0.00 | 12.78 | 1.82 |
| 110 | 60.07 | 47.51 | 79.10 | 0.00 | 9.66 | 0.00 |
| 120 | 72.07 | 57.78 | 80.28 | 0.00 | 13.84 | 0.00 |
| 130 | 84.74 | 61.49 | 72.56 | 0.00 | 19.96 | 0.47 |
| 140 | 90.40 | 67.15 | 74.28 | 0.00 | 21.76 | 1.92 |
| 150 | 96.80 | 62.45 | 64.52 | 0.00 | 28.42 | 3.54 |
| 160 | 98.74 | 59.00 | 59.76 | 0.00 | 28.97 | 6.09 |

In Table 4 below there is set out a summary of the results from HMF-DFF oxidation screening in flow using the following parameters:

1 mL HMF (5 mg/mL), H₂O, K-OMS-2/Celite, 80 bar Air, 100-160° C., 0.5 mL/min, 2 min (using one catalyst cartridge).

TABLE 4

| | HMF | DFF | DFF | | | |
|---|---|---|---|---|---|---|
| T | conversion | yield | selectivity | HMFCA | FFCA | FDCA |
| [° C.] | [%] | [%] | [%] | yield [%] | yield [%] | yield [%] |
| 100 | 32.31 | 19.17 | 59.33 | 0.00 | 1.04 | 0.00 |
| 110 | 42.28 | 30.14 | 71.28 | 0.00 | 2.37 | 0.00 |
| 120 | 54.06 | 39.36 | 72.81 | 0.00 | 4.26 | 0.00 |
| 130 | 68.06 | 48.24 | 70.87 | 0.00 | 7.39 | 0.00 |
| 140 | 78.14 | 57.02 | 72.98 | 0.00 | 9.70 | 0.00 |
| 150 | 82.29 | 61.83 | 75.14 | 0.00 | 9.06 | 0.00 |
| 160 | 84.97 | 63.69 | 74.96 | 0.00 | 10.52 | 0.00 |

In Table 5 below there is set out a summary of the results from HMF-DFF oxidation, screening in flow using the following parameters:

1 mL HMF (5 mg/mL), H₂O, 2× K-OMS-2/Celite, 80 bar Air, 100-160° C., 0.5 mL/min, 4 min (using two catalyst cartridge).

TABLE 5

| | HMF | DFF | DFF | | | |
|---|---|---|---|---|---|---|
| T | conversion | yield | selectivity | HMFCA | FFCA | FDCA |
| [° C.] | [%] | [%] | [%] | yield [%] | yield [%] | yield [%] |
| 100 | 60.53 | 36.24 | 60.30 | 0.00 | 19.86 | 0.00 |
| 110 | 64.16 | 44.51 | 69.39 | 0.00 | 10.01 | 0.00 |
| 120 | 76.13 | 52.80 | 69.37 | 0.00 | 12.93 | 0.00 |
| 130 | 85.77 | 59.16 | 68.97 | 0.00 | 16.53 | 0.00 |
| 140 | 92.83 | 61.12 | 65.85 | 0.00 | 20.26 | 0.00 |
| 150 | 95.93 | 65.46 | 68.24 | 0.00 | 19.95 | 0.00 |
| 160 | 95.18 | 66.61 | 69.98 | 0.00 | 17.45 | 0.00 |

From Tables 2 to 5 it is evident that under the given conditions a high DFF yields and a high DFF selectivity may be achieved. The yield in average is increasing with increasing temperature. A double portion of the catalyst does not result in great differences, nor does a pressure of 80 bar compared with a pressure of 10 bar.

A temperature yielding DFF in a range of approx. 50 to 70% related to the starting material HMF is in the range from approx. 100 to 160° C., e.g. 120° C. to 160° C., e.g. 140 to 160° C.

### EXAMPLE 3

Oxidation of HMF to Obtain FFCA
Reactant HMF (5 mg/mL) in water
Base additive Na₂CO₃ (2 equiv. based on HMF, premixed with HMF solution)
Catalyst 10% Pt/C (280 mg 10% Pt/C+20 mg Celite 545)
Oxidant synthetic air
Reactor System ThalesNano X-Cube, pump flow rate: 0.5mL/min, residence time: 2 min

Each CatCart (70×4 mm) was filled first with 20 mg Celite 545 and then 280 mg 10% Pt/C were added. Fresh CatCart was used every time, when the system pressure was changed. Before each screening series, the entire reaction line was purged with H₂O (HPLC Grade), the Teflon frit of the system valve was replaced and ThalesNano X-Cube System Self-Test was performed. The initial system stabilization was always achieved using H₂O (HPLC Grade) and when the reaction parameters remained constant, the pumping of the reaction solution began, then the system was allowed to stabilize and equilibrate at the new conditions for 10 min and two samples of 1 mL each were then collected. Then the temperature was increased and the system was again allowed to stabilize (the same procedure was applied for all temperatures within the experimental series). In all the cases 40 bar difference between the system pressure and the external gas pressure was provided for good system stability. At a temperature of 100° C., an ideal compromise between substrate conversion and product selectivity regarding the product FFCA was achieved.

In Table 6 below there is set out a summary of the results from HMF-FFCA oxidation screening in flow using the following parameters:

1 mL HMF (5 mg/mL), 2 equiv. Na₂CO₃, H₂O, 10% Pt/C, 80 bar Air, 60-160° C., 0.5 mL/min, 2 min.

TABLE 6

| | | | | | | FDCA/ |
|---|---|---|---|---|---|---|
| | HMF | DFF | | | | FFCA |
| T | conversion | yield | HMFCA | FFCA | FDCA | selectivity |
| [° C.] | [%] | [%] | yield [%] | yield [%] | yield [%] | [%] |
| 60 | 99.71 | 0.32 | 0.00 | 0.00 | 57.70 | 57.87/ |
| | | | | | | 0.00 |
| 80 | 99.71 | 0.32 | 1.84 | 44.90 | 43.28 | 43.41/ |
| | | | | | | 45.03 |
| 100 | 98.61 | 0.32 | 5.90 | 60.26 | 25.42 | 25.78/ |
| | | | | | | 61.11 |
| 120 | 94.15 | 0.32 | 6.55 | 56.77 | 19.80 | 21.03/ |
| | | | | | | 60.30 |
| 140 | 92.01 | 0.32 | 7.44 | 48.01 | 15.69 | 17.06/ |
| | | | | | | 52.18 |
| 160 | 85.69 | 0.32 | 9.87 | 25.41 | 13.44 | 15.69/ |
| | | | | | | 29.65 |

From Table 6 it is evident that under the given conditions a high FFCA yield and a high FFCA selectivity may be achieved. The yield in average is increasing with increasing temperature up to approx. 120° C. A temperature yielding FFCA in a range of approx. 45 to 60% related to the starting material HMF is in the range from 60° C. to 160° C., in particular from 80 to 140° C., e.g. 100 to 120° C.

### EXAMPLE 4

Oxidation of HMF to obtain FDCA
Reactant HMF (5 mg/mL) in water
Base additive NaOH (2 equiv. based on HMF, mixed in situ with the solution of HMF via the second HPLC pump, supplied as 0.08 M solution in water) or Na₂CO₃ (2 equiv. based on HMF, premixed with HMF solution) or NaHCO₃ (4 equiv. based on HMF, premixed with HMF solution)

Catalyst 10% Pt/C (280 mg 10% Pt/C+20 mg Celite 545)

Oxidant oxygen or synthetic air

Reactor System ThalesNano X-Cube, pump flow rate: 2×0.5 mL/min (NaOH), 0.5 mL/min (Na₂CO₃), 0.5 mL/min (NaHCO₃), residence time: 1 min (NaOH), 2 min (Na₂CO₃), 2 min (NaHCO₃)

Each CatCart (70×4 mm) was filled first with 20 mg of Celite 545 and then 280 mg of 10% Pt/C were added. Fresh CatCart was used every time, when the system pressure was changed. Before each screening series, the entire reaction line was purged with H₂O (HPLC Grade), the Teflon frit of the system valve was replaced and ThalesNano X-Cube System Self-Test was performed. The initial system stabilization was always achieved using either NaOH/H₂O solution, or H₂O (HPLC grade). Using either Na₂CO₃ or NaHCO₃ as base additive, the system was stabilized while pumping only H₂O (HPLC grade), not Na₂CO₃ or NaHCO₃ aqueous solution. When the reaction parameters remained constant, the pumping of the reaction solution began, then the system was allowed to stabilize and equilibrate at the new conditions for 10 min and two samples of 1 mL each were then collected. Then the temperature was increased and the system was again allowed to stabilize (the same procedure was applied for all temperatures within the experimental series). In all the cases 40 bar difference between the system pressure and the external gas pressure was provided for good system stability.

Initial experiments were carried out using NaOH as a base. Unfortunately, treating HMF solution with NaOH solution led to immediate dark colouring of the solution, followed by precipitation of black solid material rendering the solution inapplicable in flow. To overcome this problem, in-situ mixing of HMF solution and NaOH solution was performed. However, even better results were obtained switching from NaOH solution to Na2CO3 or NaHCO3 solution.

In Table 7 below there is set out a summary of the results from HMF-FDCA oxidation screening in flow using the following parameters:

0.5 mL HMF (5 mg/mL), 0.5 mL NaOH (0.08 M), H₂O, 10% Pt/C, 40 bar O₂, 60-160° C., 0.5 mL/min×0.5 mL/min, 1 min.

TABLE 7

| | HMF | DFF | | | | FDCA |
|---|---|---|---|---|---|---|
| T | conversion | yield | HMFCA | FFCA | FDCA | selectivity |
| [° C.] | [%] | [%] | yield [%] | yield [%] | yield [%] | [%] |
| 60 | 99.71 | 0.32 | 18.97 | 6.78 | 70.98 | 71.19 |
| 80 | 99.71 | 0.32 | 14.18 | 10.48 | 77.23 | 77.46 |
| 100 | 99.64 | 0.67 | 7.30 | 18.60 | 79.41 | 79.70 |
| 120 | 99.50 | 0.81 | 2.08 | 22.28 | 78.76 | 79.16 |
| 140 | 99.43 | 0.32 | 0.36 | 25.16 | 74.14 | 74.57 |
| 160 | 99.71 | 0.32 | 23.95 | 1.28 | 68.87 | 69.07 |

In Table 8 below there is set out a summary of the results from HMF-FDCA oxidation screening in flow using the following parameters:

0.5 mL HMF (5 mg/mL), 0.5 mL NaOH (0.08 M), H2O, 10% Pt/C, 80 bar O₂, 60-160° C., 0.5 mL/min×0.5 mL/min, 1 min.

TABLE 8

| | HMF | DFF | | | | FDCA |
|---|---|---|---|---|---|---|
| T | conversion | yield | HMFCA | FFCA | FDCA | selectivity |
| [° C.] | [%] | [%] | yield [%] | yield [%] | yield [%] | [%] |
| 60 | 99.71 | 0.32 | 17.23 | 8.20 | 74.15 | 74.37 |
| 80 | 99.71 | 0.32 | 11.79 | 10.67 | 79.33 | 79.57 |
| 100 | 99.59 | 0.81 | 6.16 | 17.31 | 77.97 | 78.29 |
| 120 | 99.06 | 0.32 | 1.68 | 23.86 | 76.25 | 76.98 |
| 140 | 99.34 | 0.32 | 0.79 | 31.01 | 64.85 | 65.28 |
| 160 | 99.71 | 0.32 | 27.81 | 1.29 | 57.83 | 58.00 |

From Tables 7 and 8 it is evident that under the given conditions a high FDCA yield and a high FDCA selectivity may be achieved almost independently from the temperature. A temperature yielding FDCA in a range of approx. 60 to 80% related to the starting material HMF is in the range from 60 to 160° C., e.g. 80 to 150° C.

In Table 9 below there is set out a summary of the results from HMF-FDCA oxidation screening in flow using the following parameters:

0.5 mL HMF (5 mg/mL), 0.5 mL NaOH (0.08 M), H₂O, 10% Pt/C, 40 bar Air, 60-120° C., 0.5 mL/min×0.5 mL/min, 1 min.

TABLE 9

| | HMF | DFF | | | | FDCA |
|---|---|---|---|---|---|---|
| T | conversion | yield | HMFCA | FFCA | FDCA | selectivity |
| [° C.] | [%] | [%] | yield [%] | yield [%] | yield [%] | [%] |
| 60 | 99.71 | 0.32 | 3.66 | 14.45 | 76.84 | 77.06 |
| 80 | 99.71 | 0.32 | 8.59 | 25.31 | 65.02 | 65.21 |
| 100 | 98.00 | 0.32 | 7.12 | 27.27 | 54.09 | 55.19 |
| 120 | 83.86 | 0.32 | 13.28 | 24.25 | 31.09 | 37.06 |

From Table 9 it is evident that under the given conditions a high FDCA yield and a high FDCA selectivity may be achieved. A temperature yielding FDCA in a range of approx. 60 to 80% related to the starting material HMF is in the range from 60 to 120° C., e.g. 60 to 110° C.

In Table 10 below there is set out a summary of the results from HMF-FDCA oxidation screening in flow using the following parameters:

1 mL HMF (5 mg/mL), 2 equiv. Na₂CO₃, H₂O, 10% Pt/C, 80 bar O₂, 60-120° C., 0.5 mL/min, 2 min.

TABLE 10

| | HMF | DFF | | | | FDCA |
|---|---|---|---|---|---|---|
| T | conversion | yield | HMFCA | FFCA | FDCA | selectivity |
| [° C.] | [%] | [%] | yield [%] | yield [%] | yield [%] | [%] |
| 60 | 99.71 | 0.32 | 0.00 | 0.00 | 79.02 | 79.25 |
| 80 | 99.68 | 0.32 | 0.00 | 0.00 | 91.44 | 91.73 |
| 100 | 99.71 | 0.32 | 0.00 | 0.00 | 95.23 | 95.51 |
| 120 | 99.71 | 0.32 | 0.00 | 0.00 | 95.23 | 95.51 |

From Table 10 it is evident that a high conversion rate of HMF and high yields of FDCA with high selectivity can be achieved from approx. 50° C. to 140° C. under the given conditions, and an almost complete conversion of HMF into FDCA in a temperature range of approx. 70 to 130° C.

Carrying out the example with the same reaction setup, with the only difference in that O₂-pressure was reduced to 40 bar, the following results were achieved:

TABLE 11

| | HMF | DFF | | | | FDCA |
|---|---|---|---|---|---|---|
| T | conversion | yield | HMFCA | FFCA | FDCA | selectivity |
| [° C.] | [%] | [%] | yield [%] | yield [%] | yield [%] | [%] |
| 60 | 99.55 | 0.32 | 0.00 | 0.00 | 78.62 | 78.97 |
| 80 | 99.55 | 0.32 | 11.45 | 0.00 | 68.62 | 68.94 |
| 100 | 99.51 | 0.32 | 13.96 | 0.00 | 58.15 | 58.43 |
| 120 | 99.42 | 0.32 | 12.20 | 0.00 | 49.36 | 49.65 |

Table 11 shows that with lower oxygen pressure, both FDCA yield and selectivity are decreased especially with higher temperature. This is apparently due to catalyst deactivation.

In Table 12 below there is set out a summary of the results from HMF-FDCA oxidation screening in flow using the following parameters:

1 mL HMF (5 mg/mL), 4 equiv. NaHCO₃, H₂O, 10% Pt/C, 80 bar O₂, 60-120° C., 0.5 mL/min, 2 min.

TABLE 12

| | HMF | DFF | | | | FDCA |
|---|---|---|---|---|---|---|
| T | conversion | yield | HMFCA | FFCA | FDCA | selectivity |
| [° C.] | [%] | [%] | yield [%] | yield [%] | yield [%] | [%] |
| 60 | 99.71 | 0.32 | 0.00 | 0.00 | 73.46 | 73.67 |
| 80 | 99.71 | 0.32 | 0.00 | 7.51 | 87.82 | 88.08 |
| 100 | 99.71 | 0.32 | 0.00 | 2.07 | 90.33 | 90.59 |
| 120 | 99.71 | 0.32 | 0.00 | 0.00 | 96.46 | 96.74 |

From Table 12 it is evident that a high conversion rate of HMF and high yields of FDCA with high selectivity can be achieved from approx. 50° C. to 140° C. under the given conditions, and an almost complete conversion of HMF into FDCA at temperatures above 100° C., e.g. of approx. 110° C. to 130° C.

Again, carrying out this example with the same reaction setup, with the only difference in that O₂-pressure was reduced to 40 bar, the following results were achieved:

TABLE 13

| | HMF | DFF | | | | FDCA |
|---|---|---|---|---|---|---|
| T | conversion | yield | HMFCA | FFCA | FDCA | selectivity |
| [° C.] | [%] | [%] | yield [%] | yield [%] | yield [%] | [%] |
| 60 | 99.55 | 0.32 | 0.00 | 0.00 | 66.74 | 67.04 |
| 80 | 99.55 | 0.32 | 0.00 | 5.95 | 84.79 | 85.17 |
| 100 | 99.55 | 0.32 | 0.00 | 0.00 | 82.56 | 82.94 |
| 120 | 99.55 | 0.32 | 0.00 | 0.00 | 32.14 | 32.29 |

Again, according to Table 13, with lower oxygen pressure, both FDCA yield and selectivity are decreased especially with higher temperature due to catalyst deactivation.

Thus, the above examples show that especially HMF oxidation to FDCA, employing alkali carbonates or bicarbonates as co-catalyst and employing higher oxygen pressure, yields very good results at only 2 minutes of residence time.

### EXAMPLE 5

Oxidation of HMF to Obtain FDCA Employing Water as a Solvent and NMP as Co-Solvent:

In this example, an artificial stream enriched with HMF, resembling a stream resulting from a previous dehydration of a sugar, was used as the starting material.
Artificial stream solution: 5 mg/mL HMF,
   ratio of HMF: NMP=4.7 wt %: 95.3 wt %
Base additive: NaHCO₃, 4 equiv based on HMF
Solvent: H₂O added to the artificial stream solution up to 1 mL, the NMP of the artificial stream solution acting as co-solvent
Catalyst: 10% Pt/C/Celite 545 (280 mg/20 mg)
Oxidant: O₂, pressure: 80 bar
Temperature: 60° C., 80° C., 100° C., 120° C., 140° C., 160° C.
Flow rate: 0.5 mL/min
Residence time: 2 min

The reaction was carried out in accordance with the description of Example 4 above.

In Table 14 below there is set out a summary of the results from HMF-FDCA oxidation screening in flow using the following parameters:

1 mL HMF (5 mg/mL), 4 equiv. NaHCO₃, H₂O/NMP, 10% Pt/C, 80 bar O₂, 60-160° C., 0.5 mL/min, 2 min.

TABLE 14

| | HMF | DFF | | | | FDCA |
|---|---|---|---|---|---|---|
| T | conversion | yield | HMFCA | FFCA | FDCA | selectivity |
| [° C.] | [%] | [%] | yield [%] | yield [%] | yield [%] | [%] |
| 60 | 99.55 | 0.32 | 2.25 | 38.75 | 43.52 | 43.72 |
| 80 | 99.55 | 0.32 | 0.49 | 39.55 | 59.17 | 59.43 |
| 100 | 99.15 | 0.32 | 0.00 | 19.03 | 77.42 | 78.09 |
| 120 | 99.55 | 0.32 | 0.00 | 6.19 | 90.53 | 90.94 |
| 140 | 99.55 | 0.32 | 0.00 | 1.61 | 92.13 | 92.55 |
| 160 | 99.55 | 0.32 | 0.00 | 0.72 | 80.72 | 81.09 |

From Table 14 above it becomes apparent that also based on a product stream containing NMP, good results in FDCA yield and FDCA selectivity can be obtained. The best results however, are obtained at slightly higher temperatures, such as 120° C. to 160° C.

### EXAMPLE 6

Oxidation of HMF to Obtain FDCA from a Raw Product Stream of a Preceding Sugar Dehydration Step:

A product stream obtained via dehydration of fructose with NMP as solvent, as disclosed in WO 2014/033289, was treated under the same conditions as disclosed in example 5 above.

Again, the ratio of HMF to NMP in this product stream was

HMF: NMP=4.7 wt %: 95.3 wt %.

This raw stream was pretreated before oxidation as follows:

(i) real stream dilution with pure water to the desired HMF concentration of 5 mg/mL;

(ii) centrifugation in order to separate any black tar formed during the preparation of the stream;

(iii) filtration through a filter paper;

(iv) passing the resulting solution through a packed-bed cartridge filled with activated charcoal.

In Table 15 below there is set out a summary of the results from HMF-FDCA oxidation screening in flow using the following parameters:

1 mL HMF (5 mg/mL), 4 equiv. NaHCO3, H₂O/NMP, 10% Pt/C, 80 bar O₂, 60-160° C., 0.5 mL/min, 2 min.

TABLE 15

| | HMF | DFF | | | | FDCA |
|---|---|---|---|---|---|---|
| T | conversion | yield | HMFCA | FFCA | FDCA | selectivity |
| [° C.] | [%] | [%] | yield [%] | yield [%] | yield [%] | [%] |
| 60 | 99.55 | 0.31 | 6.75 | 50.33 | 10.75 | 10.79 |
| 80 | 98.73 | 0.31 | 6.57 | 72.46 | 10.97 | 11.11 |
| 100 | 98.44 | 0.31 | 4.13 | 71.62 | 18.16 | 18.45 |
| 120 | 98.27 | 0.31 | 1.11 | 61.42 | 35.02 | 35.64 |
| 140 | 98.74 | 0.31 | 0.00 | 32.69 | 64.64 | 65.47 |
| 160 | 99.55 | 0.31 | 0.00 | 8.57 | 87.01 | 87.40 |

Table 15 shows that—although the results are slightly worse than those of an artificial stream as per Example 5—acceptable results in FDCA yield and selectivity can be obtained, again especially at higher temperatures such as from 140° C. to 160° C., without the need of prior removal of NMP from the product stream.

1. A process for the selective production of oxidized furan derivatives starting from 5-hydroxymethyl-2-furfural of formula 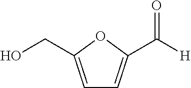
in the presence of a solvent, an oxidation agent, a catalyst, and optionally a base and/or a co-solvent, comprising:
carrying out the oxidation process continuously in flow,
providing means for varying reaction parameters,
the solvent using during the oxidation process is water and
a dipolar aprotic solvent is present as a co-solvent.

2. The process according to claim 1, wherein N-methylpyrrolidone is present as a co-solvent.

3. The process according to claim 1, wherein reaction parameters are temperature, pressure, oxidation agent, and/or catalyst.

4. The process according to claim 1, wherein said oxidized furan derivative comprises at least one aldehyde group and/or at least one carboxylic acid group.

5. The process according to claim 4, wherein said oxidized furan derivative is selected from:

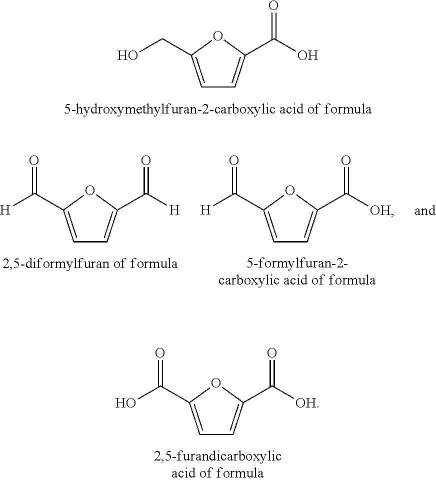

6. The process according to any one of claims 1 to 5, characterized in that the reaction temperature is from 50° C. to 180° C., in particular from 60° C. to 160° C.

7. The process according to claim 6, wherein the reaction temperature for the production of
5-hydroxymethylfuran-2-carboxylic acid is from 60° C. to 120° C., in particular from 80° C. to 120° C., in particular from 100 to 120° C.;
2,5-diformylfuran is from 100 to 160° C., in particular from 120-160° C., in particular from 140° C. to 160° C.;
5-formylfuran-2-carboxylic acid is from 60° C. to 160° C., in particular from 80° C. to 140° C., in particular from 100° C. to 120° C.;
2,5-furandicarboxylic acid is from 60° C. to 160° C., in particular from 60° C. to 120° C., in particular from 80° C. to 120° C.

8. The process according to claim 1, wherein the oxidation agent is compressed oxygen or compressed air.

9. The process according to claim 1, wherein the working pressure is from 5 bar to 100 bar, in particular from 10 bar to 80 bar.

10. The process according to claim 1, wherein the catalyst used to obtain
2,5-diformylfuran is K-OMS-2;
5-hydroxymethylfuran-2-carboxylic acid, 5-formylfuran-2-carboxylic acid and 2,5-furandicarboxylic is platinum on activated charcoal.

11. The process according to claim 1, wherein for the production of 5-hydroxymethylfuran-2-carboxylic acid, 5-formylfuran-2-carboxylic acid and 2,5-furandicarboxylic acid a base is used as a co-catalyst.

12. The process according to claim 1, wherein the base is a hydroxide, a carbonate or a bicarbonate, in particular an alkali hydroxide, an alkali carbonate or an alkali bicarbonate, in particular sodium hydroxide, sodium carbonate or sodium bicarbonate.

13. The process according to claim 1, wherein a stream enriched with 5-hydroxymethyl-2-furfural from previous dehydration reactions, in particular dehydrations of sugars, is employed as a starting material.

14. The process according to claim 1 for the selective production of 2,5-furandicarboxylic acid starting from 5-hydroxymethyl-2-furfural, characterized by the combination of the following features:
a base selected from the group of carbonates and bicarbonates, in particular sodium carbonate and/or sodium bicarbonate is used as a co-catalyst
the working pressure is from 80 to 100 bar.

15. The process according to claim 14, characterized in that the temperature is from 120° C. to 160° C., preferably from 140° C. to 160° C.

16. The process according to claims 14, wherein platinum on activated charcoal is used as the catalyst.
